(19) United States
(12) Patent Application Publication
(10) Pub. No.: US 2005/0209289 A1
(43) Pub. Date: Sep. 22, 2005

(54) Method for vascular dysregulation

(76) Inventors: Matthew T. Talarico, Morris, IL (US); Anna M. Talarico, Morris, IL (US)

Correspondence Address: Lesavich High-Tech Law Group, P.C.;Suite 325, 39 S. LaSalle Street, Chicago, IL, 60603, US

(21) Appl. No.: 11/081,571

(22) Filed: Mar. 16, 2005

Related U.S. Application Data
(60) Provisional application No. 60/553,485, filed on Mar. 16, 2004.

(51) Int. Cl. A61K 31/455; G06F 17/60; A61B 5/00

(52) U.S. Cl. 514/355; 600/300; 705/3

(57) ABSTRACT

A method for vascular dysregulation. The method includes treating a medical condition or improving a number of chronic medical conditions (e.g., asthma, etc.) by looking for certain common medical symptoms associated with the chronic medical conditions such as headaches or sinus problems and eliminating known environmental triggers, eliminating known medical triggers and adjusting patient's current medication profile and to include a calcium channel blocker, a nasal anticholinergic or an antiemetic to reduce or eliminate the common medical symptoms, thereby improving the chronic medical condition.

### CROSS REFERENCES TO RELATED APPLICATIONS

This application claims priority from U.S. Provisional Patent Application 60/553,485, filed Mar. 16, 2004, the contents of which are incorporated by reference.

### FIELD OF THE INVENTION

This invention relates to medical diagnosis and medical treatments. More specifically, it relates to a method for vascular dysregulation.

### BACKGROUND OF THE INVENTION

“Acute sinusitis” is a medical condition that affects a large number of people. The American Academy of Otolaryngology describes acute sinusitis as, “an infection of the sinus cavities caused by bacteria. It is commonly believed to be preceded by a cold, allergy attack, or irritation by environmental pollutants.”

When a patient breathes in through his/her nose, the sinuses act like a filter, which creates mucus. If a patient's sinus pathways do not properly drain, the sinuses can become “diseased.” This typically results in difficulty breathing through the nose, facial pain, and blockage of the sinuses. Acute sinusitis is a condition that will typically last more than ten days and could also cause a discharge of thick yellow/green mucus. With standard medical methods, there is no successful cure or preventive therapy for acute sinusitis.

Many of the same patients typically return over and over again with acute sinusitis. The normally accepted standard of care treatment is to treat the patient with antibiotics, decongestants, nasal sprays or even nasal surgery. However, these treatments typically provide only temporary relief from the symptoms.

There are other acute and chronic medical conditions that are also hard to treat such as asthma. In many instances, patients with these chronic medical conditions have several symptoms in common, such as headaches, etc.

Thus, it is desirable to provide a better method for treating acute sinusitis, asthma and other chronic medical problems by recognizing and treating common symptoms associated with such chronic medical problems. Also, it is desirable to provide novel treatment protocols and pathways that may also shed significant insight into previously unrecognized contributing factors and pathways for many chronic conditions. This could lead to significant prevention of many medical conditions currently not well prevented with current medical practices.

### SUMMARY OF THE INVENTION

In accordance with preferred embodiments of the present invention, some of the problems associated with treating acute sinusitis and other chronic medical conditions are overcome. A method for vascular dysregulation is presented.

The method includes treating a medical condition or improving a number of chronic medical conditions (e.g., asthma, etc.) by looking for certain common medical symptoms associated with the chronic medical conditions such as headaches or sinus problems and eliminating known environmental triggers, eliminating known medical triggers and adjusting patient's current medication profile and to possibly include a calcium channel blocker, a nasal anticholinergic and/or an antiemetic to reduce or eliminate the common medical symptoms and improve the chronic medical condition.

The foregoing and other features and advantages of preferred embodiments of the present invention will be more readily apparent from the following detailed description. The detailed description proceeds with references to the accompanying drawings.

### BRIEF DESCRIPTION OF THE DRAWINGS

Preferred embodiments of the present invention are described with reference to the following drawings, wherein:

FIG. 1A is block diagram illustrating an exemplary medical trigger continuum;

FIG. 1B is a block diagram illustrating an exemplary medical trigger continuum for headaches;

FIG. 2 is a flow diagram illustrating a Method 34 for treating medical conditions with vascular dysregulation.

FIG. 3 is a flow diagram illustrating a method for treating medical conditions with vascular dysregulation; and

FIG. 4 is flow diagram illustrating a method for altering medical thresholds for medical symptoms with vascular dysregulation.

### DETAILED DESCRIPTION OF PREFERRED EMBODIMENTS

VASCULAR DYSREGULATION

“Vascular dysregulation” refers to a physiologic state of hypersensitivity with resultant maladaptive signs and symptoms. Stated another way, it is a vascular sensitivity and response similar in nature to an “allergy” —but with a completely different physiologic pathway. For example, one vascular dysregulation pathway was discovered by linking many frequent and chronic diseases to a single common symptom, a headache. The same environmental and medical triggers and medical treatments that cause and are used to treat headaches play a role in many other, previously unlinked, chronic illnesses, including acute sinusitis, asthma, chronic rhinitis and others.

Vascular dysregulation pathways are typically overlooked physiological pathways in the human body. Vascular dysregulation pathways are a complex interplay of vasospasm and vasodilation of blood vessels, associated with inflammation and ultimately secondary to a hypersensitivity to a multitude of different environmental, dietary and medical factors. Current focus on histamine and IgE related allergies are limiting the ability to treat asthma and other allergy related conditions adequately. Acknowledging other parallel vascular dysregulation pathways provide unprecedented control over many chronic medical conditions.

Since 1980 there has been about a 100% increase in annual Asthma related deaths in the United States. Asthma is on the rise because of the increase in certain dietary triggers, most importantly, caffeine and aspartame and monosodium glutamate. There also has been huge increase in the amounts of yogurt products and well as air-fresheners and scented candles over the past few years. This causes extreme inflammation and hypersensitivity in a large number of people.

Asthma is very bad around most big cities. For example, in Chicago, Ill. because of extreme weather changes and barometric pressure changes, asthma cases are as bad as anywhere in the country. A current theory blames the concentration of concrete dust in Chicago for the large number of asthma cases. However, vascular dysregulation has illustrated that a large number of asthma cases are directly due to vasospasm and vasodilation of blood vessels caused by different types of triggers including dietary, environmental medical and other triggers.

Children are facing growing health problems including obesity, diabetes, depression. Childhood asthma currently accounts for about 10% of all emergency room visits and about 10% of all pediatric hospitalizations. Several studies have shown that appropriate treatment of asthma with oral antihistamines or intranasal cortlcosteroids can aid in the management of asthma. However, most experts feel that such asthma treatments ultimately improve nasal inflammation and reduce nasal secretions. Vascular dysregulation provides the same end result without associated side affects.

The rise in Attention Deficit Disorder (ADD)/Attention-Deficit Hyperactivity Disorder (ADHD) can also be explained with vascular dysregulation including factors such as increased nasal congestion and increasing, cumulative trigger levels that cause poor sleep quality and resultant fatigue, hyperactivity and behavioral problems. There also appears to be a strong connection between barometric pressure changes and syncope (i.e., fainting) and Sudden Infant Death Syndrome (SIDS) and other conditions that can be addressed with vascular dysregulation.

It is estimated that that about 12% of the worldwide population suffers from fibromyalgia. As is known in the medical art, fibromyalgia is a chronic disorder characterized by widespread musculoskeletal pain, fatigue, and multiple tender points that occurs in precise, localized areas, particularly in the neck, spine, shoulders, and hips. Fibromyalgia also causes sleep disturbances, morning stiffness, irritable bowel syndrome, anxiety, and other chronic symptoms. Stress and inability to cope with life are typically and incorrectly blamed for fibromyalgia. However, it has been determined that vasospasm and vasodilation of blood vessels contribute to fibromyalgia and vascular dysregulation can be used to improve fibromyalgia symptoms in many patients.

MEDICAL TRIGGERS

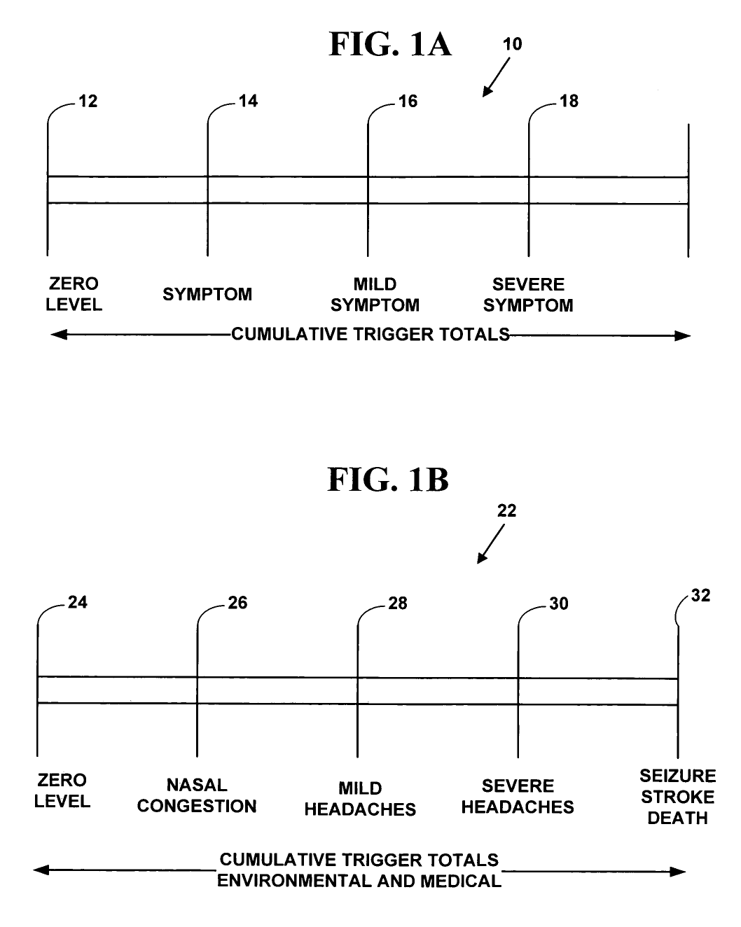

FIG. 1A is block diagram illustrating an exemplary medical trigger continuum 10. At one end of the continuum 12 is a zero value. At a first level 14, a medical symptom first appears. At a second level 16, the medical symptom becomes a mild medical symptom. At a third level 18, the medical symptom becomes severe.

Medical triggers are cumulative in a patient and vary from patient to patient. A third level 16 when a medical symptom becomes severe in a first patient may not even appear at a first level 12 in a second patient. However, certain thresholds of cumulative medical triggers when reached always manifest in one or more types of medical symptoms. Thus, it is desirable to push the medical triggers levels back along the continuum as close to the zero value level 12 as possible.

In the present invention, it has been clinically determined that the triggers include at least a medical trigger and an environment trigger that are closely interrelated.

As another example, based on recent clinical studies it was determined with clinical data that a same group of patients were returning over and over again with acute sinusitis and were being treated with antibiotics. Many of these same patients also had a history of headaches—quite often migraines. The medical literature suggests that most “sinus” headaches are migraine headaches.

FIG. 1B is block diagram illustrating an exemplary medical trigger continuum for headaches 22.

A zero level is illustrated at 24. A symptom of nasal congestion very commonly appears at a first level 26. A symptom of mild headache appears at a second level 28. A symptom of severe headache appears at a third level 30. A symptom of seizure, stroke and/or death may appear at a fourth level 32. Thus, it is desirable to lower a cumulative medical trigger level as close to the zero level 24 as possible for a patient using vascular dysregulation.

As is known in the medical arts, current medical practice is to treat acute sinusitis with antibiotics. However, a number of clinical studies have failed to show benefit for treating acute sinusitis with antibiotics over placebo. It was determined that treating these same patients' “migraine” headaches with using the vascular dysregulation pathway via aggressive environmental and trigger avoidance and prophylactic therapy significantly decreased their sinus disease, nasal congestion as well as the symptoms of other chronic diseases as explained below. Thus, treatment using the vascular dysregulation pathway helped lower cumulative medical triggers in the patients and push the cumulative medical triggers back towards the zero level 24. As is known in the medical arts “prophylactic therapy” is a medical therapy that prevents or slows the course of an illness or disease via doctor recommendation among several preventative alternatives. Thus, vascular dsyregulation can be used to treat many previously unlinked illnesses.

TREATING MEDICAL CONDITIONS WITH VASCULAR DYSREGULATION

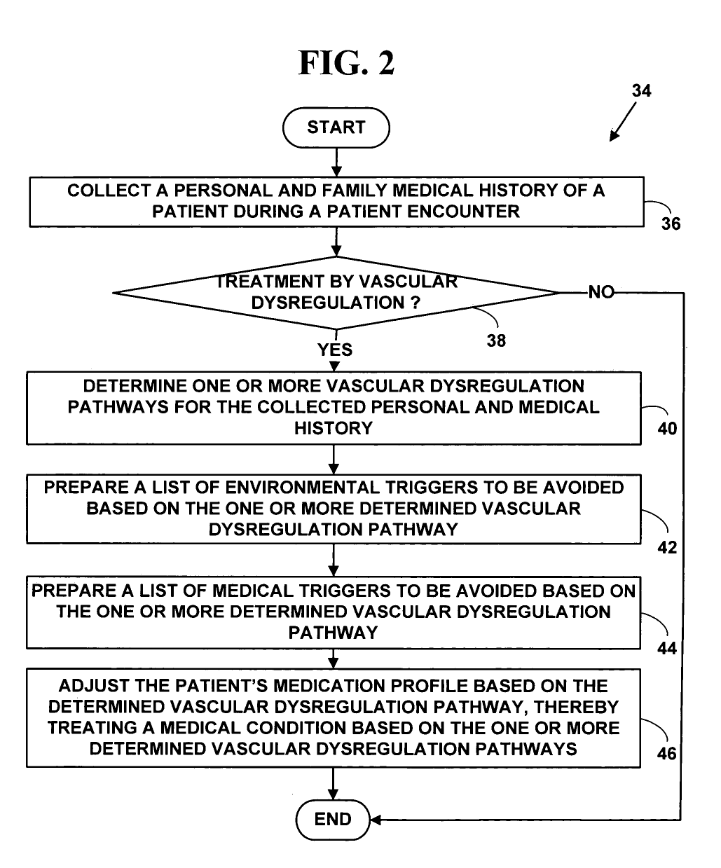

FIG. 2 is a flow diagram illustrating a Method 34 for treating medical conditions with vascular dysregulation. At Step 36, a personal and family medical history of a patient is collected during a patient encounter. At Step 38, a test is conducted to determine whether the patient has one or more symptoms that allow treatment by vascular dysregulation based on the personal and medical history collected during the patient encounter. If the patient has the one or more symptoms that would allow treatment by vascular dysregulation, at Step 40, one or more vascular dysregulation pathways are determined. At Step 42, a list of environmental triggers for the one or more determined dysregulation pathways to be avoided is prepared for the patient. At Step 44, a list of medical triggers for the one or more determined dysregulation pathway to be avoided is prepared for the patient. At Step 46, the patient's current medication profile, if any, is adjusted for the one or more determined dysregulation pathways, thereby treating the medical condition via the one or more determined dysregulation pathways.

Method 32 is illustrated with an exemplary embodiment. However the present invention is not limited to this embodiment and other embodiments can also be used to practice the invention.

In such an exemplary embodiment at Step 34, a personal and family medical history of a patient is collected during a patient encounter such as an office visit, emergency room visit or routine physical. For example the patient may be asked if they suffer from nasal congestion, headaches, fatigue, shortness of breath, etc. As an example, to illustrate Method 34, clinical data from an actual patient encounter is used.

Patient-A is a 28 year old female with history of migraine headaches, nasal congestion and chronic asthma which requires use of a combination inhaler. Patient-A drinks several cups of coffee and diet sodas daily, used scented deodorants, scented candles and air fresheners regularly.

At Step 36, a test is conducted to determine whether Patient-A has one or more symptoms that allow treatment by vascular dysregulation based on the personal and medical history collected during the patient encounter. In one embodiment, patients expected to show good clinical response from treatment for vascular dysregulation will likely share at least some of the following symptoms/signs/illnesses illustrated in Table 1. However, the present invention is not limited to these symptoms/signs/illnesses illustrated in Table 1 and more, fewer or other symptom/signs/illnesses can also be used to practice the invention.

TABLE 1
Migraine headaches (personal history or relative)
“Sinus” headaches
Asthma
Chronic rhinitis, post-nasal drip, infraorbital ecchymosis, chronic
mouth-breathing
History of frequent ear &/or sinus infections, recurrent sore throats
History of tonsillectomy, adenoidectomy, endoscopic sinus surgery
and/or tympanostomy tube placement
Cold distal extremities (Raynaud's)
History of anxiety &/or panic attacks
Type A personality, bright, high achieving
Chronic fatigue, unrefreshing sleep
Motion sickness (especially if riding in backseat or attempting to
read in car)
Feeling physically ill/sick in stores or workplaces with fluorescent lighting
Headaches or other symptoms that correlate to weather changes
Hypersensitivity with any of the five senses
Irritable bowel syndrome (IBS)
Unexplained dizziness or other neurologic complaints (with other serious
illnesses ruled out)
Premenstrual Syndrome (PMS)
Depression
ADD/ADHD

Patient-A has at least three symptoms/signs/illnesses listed in Table 1, so Patient-A is a good candidate for treatment by vascular dysregulation.

If the patient has one or more symptoms illustrated in Table 1 that would allow treatment by vascular dysregulation, at Step 40 one or more vascular dysregulation pathways are determined based on the one or more symptoms.

Table 2 illustrates exemplary vascular dysregulation pathways. However, the present invention is not limited to the exemplary vascular dysreguation pathways and more, fewer of other vascular dysregulation pathways can also be used to practice the invention.

TABLE 2
Nasal congestion: (a) hypersensitive reaction → (b) vascular hypersensitivity → to (a).
Headache: (a) nasal congestion → (b) irritated membranes and blood vessels → to (a).
Asthma: (a) membrane hypersensitivity → (b) airway resistance → (c) headache → (d) nasal
congestion → to (a).
Fatigue: (a) nasal congestion → (b) poor sleep quality → (c) fatigue → (d) increased
stimulant usage (e.g., caffeine, etc). → to (a).
Hypertension: (a) nasal congestion → (b) poor sleep quality -> (c) increased adrenaline →
(d) increased vessel pressure → to (a).
Obesity: (a) airway resistance → (b) poor physical performance → (c) withdrawal from
physical activities → (d) more sedentary activities → to (a).
Attention Deficit Disorder (ADD)/Attention-Deficit Hyperactivity Disorder (ADHD): (a) nasal
congestion -> (a) poor sleep quality → (b) fatigue → (c) hyperactivity → (d) poor attention
and focus → to (a).
Substance Abuse/Addiction: (a) chronic nasal congestion or headaches → (b) poor sleep
quality → (c) anxiety, to (b) and → self-treatment with drugs, alcohol, tobacco, sex, food, etc.
→ to (a).
Anxiety: nasal congestion -> (a) poor sleep quality → unsettled feelings → prescribed anti-
depressants → to (a).
Depression: nasal congestion ->(a) poor sleep quality → severe unsettled feelings →
prescribed anti-depressants → to (a).
Pulmonary: (a) hypersensitivity → (b) nasal congestion → (c) upper airway resistance → (d)
shortness of breath → (e) rhinitis and post-nasal drip → coughing → to (a).
Infections: (a) acute sinusitis → (b) acute otitis media → (c) eustachian tube dysfunction →
to (a).
Common Cold: (a) upper airway inflammation → (b) sore throat → (c) upper airway
resistance → (d) coughing → to (a).
Downs Syndrome: (a) nasal congestion and inner ear obstruction → (b) hearing problems →
(c) slowed learning → to (a).
Colic: (a) hypersentivity reaction → (b) irritability → (c) abdominal discomfort → to (a).
Sleep Apnea: nasal congestion -> (a) upper airway resistance → (b) obstructed airflow →
poor sleep quality and hypoxia complications→ to (a).
Insomnia: (a) anxiety → (b) adrenaline → (c) poor sleep quality → to (a).
Snoring: nasal congestion -> (a) upper airway resistance → (b) cardiac resistance → (c)
poor sleep quality → to (a).
Gastroesophageal Reflux Disease (GERD): chronic congestion -> snoring -> negative
intrathoracic pressure -> to (a).
Irritable Bowl Syndrome (IBS): (a) vascular dysregulation triggers → (c) bowel irritability → to
(a).
Raynaud's: (a) vasospastic attacks → (b) blood vessels constrict → to (a).
Sudden Infant Death Syndrome (SIDS): (a) changes in barometric pressure → (b) upper
airway resistance → (c) cardiac complication (?arrhythmia) → death.
Stroke: (a) blood vessels constrict or obstructed → (b) decreased or blocked blood vessel
flow.
Seizures: (a) irritated membranes and blood vessels → (b) disturbed neuronal activity →
(c) (d) convulsions → to (a).
Chronic neck pain or stiffness: (a) irritated membranes and blood vessels → (b) fatigue →
(c) headache → (d) poor sleep quality → to (a).
Premenstrual Syndrome (PMS): (a) monthly hormonal changes → increase in nasal
congestion -> poor sleep quality -> (b) fatigue → (c) moodiness → (d) irritability → to (a).
Chronic Fatigue Syndrome: nasal congestion -> (a) poor sleep quality → (b) fatigue → (c) →
to (a).
Fibromyglaia: nasal congestion -> poor sleep quality -> loss of “self-healing” benefit of deep
stages of sleep → to (a).
Chronic Lower Back Pain: (a) irritated membranes and blood vessels → nasal congestion ->
poor sleep quality -> loss of “self-healing” benefit of deep stages of sleep → to (a).
Tourette's Syndrome: (a) poor sleep quality → (b) increased anxiety → (c) increased tics to
(a).
Panic Disorder: (a) poor sleep quality → (b) unsettled feelings → (c) prescribed anti-
depressants → to (a).
Dementia (Alzheimer's or vascular): (a) poor sleep quality → (b) fatigue → (c) poor attention
and focus → to (a).

Patient-A has the vascular dysregulation asthma pathway as well as the vascular dysregulation headache and nasal congestion pathways illustrated in Table 2.

Returning to FIG. 2 at Step 42, a list of environmental triggers to be avoided is prepared for the patient based on the one or more determined vascular dysregulation pathways.

Table 3 illustrates exemplary environmental triggers. However, the present invention is not limited to the environmental triggers illustrated in Table 3, and more, fewer of other environmental triggers can also be used to practice the invention.

TABLE 3

Fluorescent lighting
Bright lights
Loud noises
Long periods of travel in airplanes or automobiles
Missing meals
Cigar/Cigarette smoke
After Shave
Perfume
Scented Deodorants
Air Fresheners
Scented Candles
Variations in Sleep Cycles
Living Space environment changes such as barometric pressure,
temperature, humidity, airflow

For Patient-A at Step 42, the list of environmental triggers to be avoided for include cigar/cigarette smoke, after shave, perfume, scented deodorants, air fresheners, scented candles, and low humidity in the living environment. Eliminating or reducing these environmental triggers help reduce membrane hypersensitivity that contributes to airway resistance, headaches and nasal congestion.

At Step 44 a list of medical triggers to be avoided is prepared for the patient based on the one or more determined vascular dysregulation pathways.

Table 4 illustrates exemplary medical triggers. However, the present invention is not limited to the medical triggers illustrated in Table 4, and more, fewer of other medical triggers can also be used to practice the invention.

TABLE 4

Caffeine
Aspartame and other artificial sweeteners
Monosodium Glutamate (MSG)
Herbal Supplements, Over the counter medications, Prescribed
medications and inappropriate combinations thereof
Yogurt
Nut products
Other suspected dietary migraine triggers

For example, the patient may be advised via a list of dietary changes such as the elimination of common dietary migraine triggers, particularly caffeine (coffee and diet sodas) and aspartame (diet sodas), since these substances have no nutritional benefit and trigger many known allergic reactions causing vascular irritation.

At Step 46, the patient's current medication profile is adjusted. Typically multiple prescriptions medications and/or OTC medications are eliminated or significantly reduced. Additional specialized vascular dysregulation medications may then be prescribed.

For example, at least one family of prophylactic prescription medicines has also been successfully used at Step 46 for many different vascular dysregulation pathways. In one embodiment, calcium channel blockers such as verapamil are used. However, the present invention is not limited to such an embodiment and other calcium channel blockers or other calcium channel blockers with similar chemical structures and properties can also be used to practice the invention.

The family of prophylactic prescription medicines including verapamil, by itself or in combination pills containing it and other similar diltiazem products such as cardizem, which are known as “calcium channel blockers.” The prophylactic prescription medicines may also include nasal sprays and other types of solids, liquids or aerosols. Existing medications containing verapamil or novel combinations of verapmil and cardizem has been successfully used as specialized vascular dysregulation medication.

Verapamil-based medications are typically prescribed for several heart and blood pressure problems. The fast-acting brands (e.g., Calan FA and Isoptin FA) are taken for angina (i.e., chest pain due to clogged cardiac arteries), as well as irregular heartbeat and high blood pressure. The longer-acting brands (e.g., Calan SR, Isoptin SR, Verelan, and Verelan PM) are typically used only for high blood pressure. Covera-HS is prescribed for both high blood pressure and angina.

However, such drugs can also be used to reduce vascular irritation and reduce nasal congestion and the many commonly associated medical conditions.

Verapamil eases the heart's workload by slowing down the passage of nerve impulses through it, and hence the contractions of the heart muscle. This improves blood flow through the heart and throughout the body, reduces blood pressure, corrects irregular heartbeat, and helps prevent angina pain. Some doctors also prescribe verapamil to directly treat migraine headaches and asthma and to treat manic depression and panic attacks.

Cardizem, Cardizem CD, and Cardizem LA (i.e., a controlled release form of diltiazem) are used in the treatment of angina pectoris (i.e., chest pain usually caused by lack of oxygen to the heart due to clogged arteries) and chronic stable angina (i.e., caused by exertion). Cardizem CD is also used to treat high blood pressure. Another controlled release form, Cardizem SR, is used only in the treatment of high blood pressure. Cardizem, a calcium channel blocker, dilates blood vessels and slows the heart to reduce blood pressure and the pain of angina.

Doctors sometimes prescribe cardizem for loss of circulation in the fingers and toes (e.g, Raynaud's phenomenon), for involuntary movements (e.g, tardive dyskinesia), and to prevent heart attack.

In one embodiment, verapamil and cardizem are combined for use at Step 46. The combination maximizes the benefits of each and also acts to minimize the side effects of each. In another embodiment, either verapamil or cardizem is used alone, one without the other. However, the present invention is not limited to these embodiments and other embodiments can also be used to practice the invention.

Tiazac and Dilacor XR are used in the treatment of high blood pressure and chronic stable angina. They may be taken alone or combined with other blood pressure medications.

Atrovent nasal spray is also prescribed for vascular dysregulation. Atrovent effectively reduces allergic and non-allergic medical and environmental triggers. However, the present invention is not limited to such an embodiment and other bronchial spasm preventers with similar chemical structures and properties can also be used to practice the invention.

Atrovent nasal spray relieves runny nose. The 0.03% spray is used for year-round runny nose due to allergies and other causes. The 0.06% spray is prescribed for hay fever and for runny nose due to colds. Atrovent nasal spray can also be used as a slow-release, long acting formulation.

In one embodiment, Atrovent is used alone at Step 46. In another embodiment Atrovent is combined with a nasal steroid combination. In another embodiment, Atrovent is used with verapamil and/or cardizem. However, the present invention is not limited to these embodiments and other embodiments can also be used to practice the invention.

Tigan (and other related anti-emetics, like compazine) are also prescribed for vascular dysregulation. Tigan is an antiemetic that is used to prevent or lessen nausea and vomiting, and has been prescribed “off-label” for acute treatment of migraine headaches.

Tigan and other antiemetics are used to prevent or lessen nausea and vomiting. However, the present invention is not limited to such an embodiment and other antiemetics with similar chemical structures and properties can also be used to practice the invention.

Eliminating and/or reducing the environment triggers and the medical triggers, and taking a daily verapamilverapamil has eliminated Patient-A's headaches and nasal congestion by stabilizing blood vessels, reducing inflammation, and has significantly reduced Patient-A's asthmatic activity eliminating the need completely for the prophylactic “combination” inhaler. Thus, Patient'A's cumulative medical trigger threshold has been moved closer to the zero level 24 (FIG. 1B) via treatment with vascular dysregulation.

TREATING CHRONIC MEDICAL CONDITIONS WITH VASCULAR DYSREGULATION

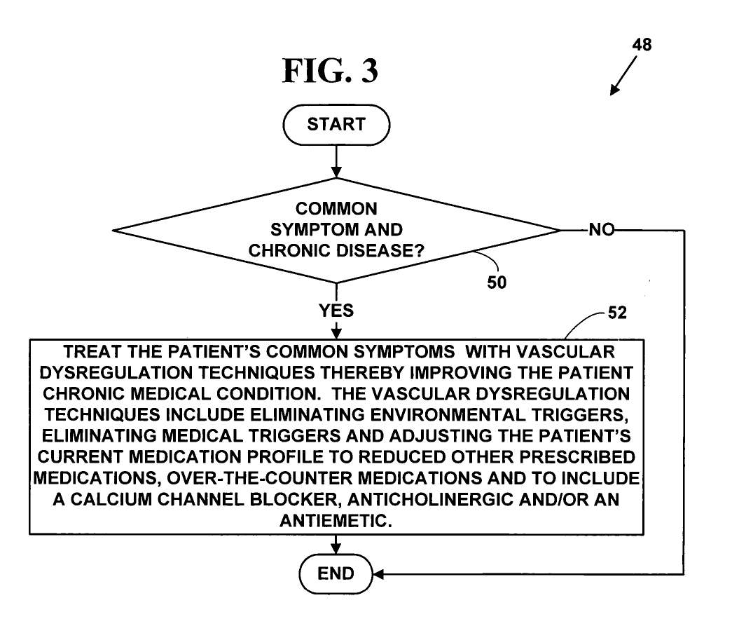

FIG. 3 is flow diagram illustrating a Method 48 for treating chronic medical conditions with vascular dysregulation. At Step 50, a test is conducted to determine whether a patient has a chronic medical condition and has one or more common symptoms based on a personal and medical history collected during a patient encounter. If the patient has a common symptom at Step 52, the patient's common symptoms are treated with vascular dysregulation techniques thereby improving the patient chronic medical condition. The vascular dysregulation techniques include eliminating environmental triggers, eliminating medical triggers and adjusting patient's current medication profile. For example, once proper identification has been made of the one or more common symptoms, at Step 52 a treatment is based on a multiple steps rather than prescribing a single therapeutic agent. The chronic medical problem is thus improved with vascular dysregulation.

Method 48 is illustrated with an exemplary embodiment. However the present invention is not limited to this embodiment and other embodiments can also be used to practice the invention. As an example, to illustrate Method 48, clinical data from an actual patient encounter is used.

In such an embodiment at Step 50 a test is conducted to determine whether a patient has a chronic medical problem, such as asthma, has one or more common symptoms, such as headaches, or sinus problems based on a personal and medical history collected during a patient encounter.

For example, Patient-B is a 52 year old male with obesity, hypertension, shortness of breath and tobacco addiction but no known cardiovascular disease. Patient-B also exhibited chronic nasal congestion, headaches and fatigue. Patient-B drank three or more cups of coffee daily. Thus, Patient-B can be treated with vascular dysregulation.

At Step 52, Patient-B's environmental triggers were adjusted to smoke outside the house and shower every night before bedtime to remove smoke particles from tobacco use. Patient-B's medical triggers were adjusted to remove caffeine. Patient-B's other prescribed medicines for hypertension and other OTC medications were eliminated, and he began use of verapamil.

Patient-B exhibited dramatic medical improvement in exercise tolerance and breathing after eliminating caffeine and all other prescription and OTC medications and beginning use of verapamil. Patient-B exhibited the dramatic medical improvement without altering tobacco use or losing weight. Thus, Patient-B's chronic medical conditions were improved with vascular dysregulation.

ALTERING MEDICAL THRESHOLD FOR MEDICAL SYMPTONS WITH VASCULAR DYSREGULATION

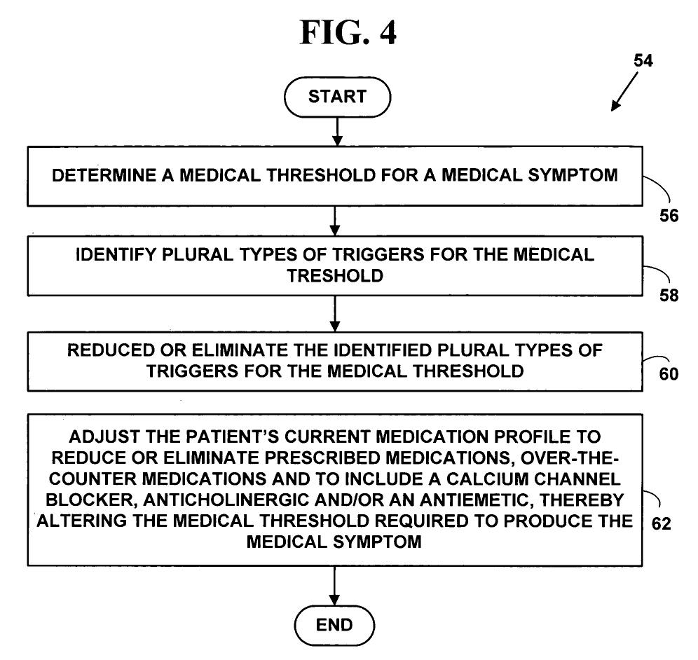

FIG. 4 is flow diagram illustrating a Method 55 for altering medical thresholds for medical symptoms with vascular dysregulation. At Step 56, one or more medical thresholds for one or more medical symptoms are determined for a patient. At Step 58, plural types of triggers for the one or more medical thresholds are identified. At Step 60, the identified plural types of triggers are reduced or eliminated. At Step 62, the patient's current medication profile is adjusted to reduce or eliminate prescribed medications, over-the-counter medications and to include a calcium channel blocker, a nasal anticholinergic or an antiemetic, thereby altering the one or more medical thresholds required to produce the one or more medical symptoms and reducing cumulative effects of the one or more medical symptoms.

Method 54 is illustrated with an exemplary embodiment. However the present invention is not limited to this embodiment and other embodiments can also be used to practice the invention. As an example, to illustrate Method 54, clinical data from an actual patient encounter is used.

At Step 56, one or more medical thresholds for one or more medical symptoms are determined for Patient-C. Patient C is a 43 year old female with recurrent sinus “infections,” migraine headaches, and chronic fatigue and was scheduled for sinus surgery. Patient-C regularly drinks coffee and diet sodas with artificial sweeteners and uses OTC sinus medicines and regularly used scented candles and air fresheners in her home. Patient-C is at her severe medical threshold for nasal congestion and is at her severe medical threshold for headaches.

In one embodiment, Step 56 includes identifying one or more vascular dysregulation pathways for medical thresholds associated with one or more medical symptoms (e.g., Table 2). However, the present invention is not limited to such an embodiment, and other embodiments can also be used to practice the invention.

At Step 58, environmental triggers including scented candles and air fresheners are identified. In addition, at Step 58, medical triggers including caffeine and aspartame are identified (e.g., Tables 3 and 4).

At Step 60, the identified environmental triggers and medical triggers were eliminated. Patient-C got rid of her scented candles and air fresheners. Patient-C also stopped drinking coffee and diet sodas.

At Step 62, Patient-C's current medication profile was adjusted to eliminate all OTC sinus medicines. Patient-C was then prescribed verpamil. Patient-C altered the medical thresholds required to produce the severe sinus symptoms and the severe headache symptoms.

Patient-C eliminated caffeine and aspartame in her diet and takes a daily verapamil. Patient-C avoided “necessary” sinus surgery and has enjoyed wonderful sinus health, no headaches and increased energy and has no longer suffered from any severe medical symptoms to date.

EXAMPLARY CLINICAL DATA

Based on actual clinical data collected, the methods described herein have been used to improve the health of a number of patients. Patients with severe, chronic sinus problems are clearer and healthier than ever before; patients with asthma are able to wean off some, if not all, of their maintenance medications; many patients chronically fatigued now have more energy than ever before; some younger patients no longer need their ADD/ADHD medications. The patients are experiencing fewer sore throats, sinus infections, asthma attacks, and hospital admissions than should be possible with standard medical practice. They need significantly fewer medications and fewer doctor visits. Some surgeries have already been avoided. With improved breathing and sleep quality, energy improves and patients become more active, lose weight and enjoy better health than ever before.

Many patients, after discovering more about their triggers and threshold, are able to wean off of their prophylactic medications or use them on an as-needed basis. Results are maintained. Success rates are dramatic when patients are selected successfully for treatment with vascular dysregulation techniques.

Exemplary Clinical Data

The names and other identifying information were eliminated and the clinical information generalized to protect the privacy of these clinical patients under Health Insurance Portability and Accountability Act (HIPAA). Use of the generalized patient information does not violate the privacy of any real individual under HIPAA or other federal or state laws.

Patient A: 28 year old female with history of chronic asthma and migraine headaches. Eliminating caffeine and aspartame, and taking a daily verapamil has significantly reduced her asthma activity and eliminated the need for her prophylactic “combination” inhaler.

Patient B: 52 year old male with obesity, hypertension, shortness of breath and tobacco addiction but no known cardiovascular disease. Also exhibited chronic nasal congestion, headaches and fatigue. Patient-B exhibited dramatic improvement in exercise tolerance and breathing after eliminating caffeine and all other prescription and OTC medications and beginning use of verapamil. Patient-B exhibited significant improvement in breathing and exercise tolerance without altering tobacco use or losing weight.

Patient C: 43 year old female with recurrent sinus “infections,” migraine headaches, and chronic fatigue and was told by the otolaryngologist that she should have required immediate sinus surgery. Patient-C eliminated caffeine and aspartame in her diet and takes a daily verapamil. She avoided “necessary” sinus surgery and has enjoyed wonderful sinus health, no headaches and increased energy, all with significantly less physician visits.

Patient D: 28 year old female with severe and chronic sinus disease, migraine headaches, asthma and clinical allergy symptoms. Treatment of traditional “allergies,” associated with its own specific pathway, was never enough to control her chronic symptoms. After several unsuccessful specialist visits, she finally obtained dramatic relief from headaches, nasal congestion and asthma after elimination of common dietary “migraine” triggers, modification of other environmental triggers and verapamil therapy. Traditional histamine-related “allergy” treatments factored into her success much less so than predicted by medicine's current approach.

Patient E: 31 year old male with a history of cold toes and fingers, migraine headaches, not only obtained warm fingers and toes off caffeine and on diltiazem, but also noted a pleasant unexpected effect: a significantly stronger singing voice—presumed secondary to decreased post-nasal drip and decreased nasal congestion.

Patient F: 32 year old female who has cough-predominant asthma notes significant coughing whenever she drinks colas with caffeine and/or aspartame. Avoidance of the identified triggers in combination with daily verapamil has significantly improved her exercise tolerance and eliminated her cough symptoms which also led to improved sleep quality.

Patient G: 55 year old male with hypertension who snored and had fatigue and drank coffee regularly. He eliminated coffee and stopped his original hypertension medication A new hypertension medication combination that includes verapamil was prescribed. He no longer snores and has significantly more energy. His blood pressure is even better controlled than before verapamil.

Patient H: 32 year old female with severe headaches and insomnia who drank coffee and diet sodas regularly. By eliminating coffee and diet sodas and taking cardizem daily, her severe headaches were eliminated and she is sleeping soundly.

Patient I: 16 year old male treated for many years for ADHD with stimulant medications suffered growth retardation as a side-effect. He was also a chronic mouth breather with poor sleep quality and poor diet. Environmental triggers and medical triggers were modified and combined with daily verapamil and he was quickly weaned off his stimulant medications for ADHD. He slept much better, his focus improved and a normal growth pattern returned. His peers even noted a dramatic improvement in his affect.

Patient J: 5 year old male with chronic nasal congestion who was excessively hyperactive and violent and did not sleep through the night. Eliminating a few environmental triggers and adding a small dose of verapamil significantly changed his behavior. He soon slept through the night and became normoactive and eliminated the chronic nasal congestion. Changes in weather's barometric pressure required different doses of verapamil to continue modified behavior.

Patient K: 31 year old caffeine-using male with anxiety disorder, headaches, Raynaud's, poor sleep quality needed prescribed antidepressant/anti-anxiety drugs. When he gave up caffeine and eliminated several environmental triggers combined with starting daily Cardizem LA, he then enjoyed significant improvement in sleep quality, no more anxiety and was able to be weaned off prescribed antidepressant/anti-anxiety drugs.

Patient L: 28 year old male who suffered moderate to severe non-suicidal depression. He could not tolerate typical prescribed antidepressant drugs. Modified several environmental and medical triggers, started Cardizem LA and noted significant improvement in moods very quickly. It has also helped relieve the majority of his vague, chronic leg pain.

Patient M: 22 year old female with asthma, chronic nasal congestion is also a caffeine user and tobacco addict. When she eliminated caffeine and took a daily dose of verapamil, it virtually eliminated the asthma and nasal congestion and dramatically reduced asthma medication. She significantly felt better and lost weight without altering tobacco use.

Patient N: 40 year old male with history of recurrent sinus infections. Altered environmental triggers combined with a daily dose of verapamil. Eliminated sinus problems and did not develop any new sinus infections.

Patient O: 40 year old female with recurrent sinus infections, headaches, nasal congestion and a caffeine user. Modified environmental and medical triggers combined with a daily dose of verapamil. Showed significant improvement in her pre and post-treatment sinus CT scan.

Patient P: 32 year old female presented to the emergency department with a severe migraine headache and complex signs including numbness and weakness on one side of her face and upper body. After receiving a small dose of verapamil and tigan orally, all symptoms dramatically resolved within 30 minutes. She was discharged from the emergency department without any further workup necessary. Environmental and medical triggers were modified and she was to continue verapamil. The migraine headaches and complex signs did not return.

Patient Q: 80 year old male with stroke like symptoms in the emergency department. Was given lverapamil and tagan orally, all symptoms were dramatically resolved within 30 minutes. The symptoms did not return with continued verapamil and tagan does.

Multiple young children (between the ages of 6 and 12) with moderate to severe asthma at baseline have weaned off multiple maintenance medications since making minor dietary adjustments. Some benefit even more by taking low doses of verapamil.

The methods described herein can be used to improve a number of chronic medical conditions (e.g., asthma, etc.) by looking for certain medical symptoms common to the chronic medical conditions such as headaches, and eliminating environmental triggers, eliminating medical triggers and adjusting patient's current medication profile to reduce or eliminate the common medical symptoms, thereby improving the chronic medical condition.

The methods described herein may significantly lower health cost and insurance costs. After applying the methods herein, patients typically require less diagnostic studies, fewer procedures and surgeries, fewer office visits and fewer hospitalizations and lower mental heath costs. The patients expenses for prescribed and OTC drugs are lower, with fewer missed work or school days resulting in more productive workers and students.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described, and more fewer or equivalent elements may be used in the block diagrams.

The claims should not be read as limited to the described order or elements unless stated to that effect. In addition, use of the term “means” in any claim is intended to invoke 35 U.S.C. §112, paragraph 6, and any claim without the word “means” is not so intended.

Therefore, all embodiments that come within the scope and spirit of the following claims and equivalents thereto are claimed as the invention.

1. A method for treating medical conditions with vascular dysregulation, comprising:
collecting a personal and family medical history of a patient during a patient encounter; and
determining whether the patient has one or more symptoms that allow treatment by vascular dysregulation based on the collected personal and medical history, and if so,
determining one or more vascular dysregulation pathways for the one or more symptoms,
preparing a list of environmental triggers for the one or more determined dysregulation pathway to be avoided,
preparing a list of medical triggers for the one or more determined dysregulation pathways to be avoided, and
adjusting the patient's current medication profile, for the one or more determined dysregulation pathways,
thereby treating or preventing the medical condition based on the one or more determined dysregulation pathways.

2. The method of claim 1 wherein the one or more vascular dysregulation pathways include a nasal congestion, headache, asthma, fatigue, hypertension, obesity, attention deficit disorder (ADD)/attention-deficit hyperactivity disorder (ADHD), substance abuse, substance addiction, anxiety, depression, panic disorder, respiratory infections, common colds, downs syndrome, colic, insomnia, snoring, sleep apnea, gastroesophageal reflux disease (GERD), Raynaud's phenomenon, sudden infant death syndrome (SIDS):, stroke, seizures, chronic neck pain, premenstrual syndrome (PMS), chronic fatigue syndrome, Tourette's syndrome, Alzheimer's, or a dementia vascular dysregulation pathway.

3. The method of claim 1 wherein the environmental triggers include fluorescent lighting, bright lights, loud noises, long periods of travel in airplanes or automobiles, missing meals, cigar or cigarette smoke, after shave lotion, perfume, scented deodorants, air fresheners scented candles, variations in sleep cycles, living space environment changes such as barometric pressure, temperature, humidity, or airflow.

4. The method of claim 1 wherein the medical triggers include caffeine, aspartame, Monosodium Glutamate (MSG), herbal supplements, over the counter medications, prescribed medications, yogurt or nut products and other commonly accepted dietary migraine triggers.

5. The method of claim 1 wherein the step of adjusting the patient's current medication profile comprises:
eliminating one or more prescribed medications;
eliminating one or more over-the-counter medications; and
including a calcium channel blocker in the patient's current medication profile.

6. The method of claim 5 wherein the calcium channel blocker includes verapamil, cardizem or tarka.

7. The method of claim 5 wherein the calcium channel blocker includes verapmil and cardizem in combination

8. The method of claim 1 wherein the step of adjusting the patient's current medication profile comprises:
eliminating one or more prescribed medications;
eliminating one or more over-the-counter medications; and
including a nasal anticholinergic in the patient's current medication profile.

9. The method of claim 8 wherein the nasal anticholinergic includes atrovent.

10. The method of claim 1 wherein the step of adjusting the patient's current medication profile comprises:
including a calcium channel blocker and a nasal anticholinergic in the patient's current medication profile.

11. The method of claim 1 wherein the step of adjusting the patient's current medication profile comprises:
eliminating one or more prescribed medications;
eliminating one or more over the counter medications; and
including an antiemetic in the patient's current medication profile.

12. The method of claim 11 wherein the antiemetic includes tigan or other antiemetics such as compazine and reglan.

13. A method for treating chronic medical conditions by vascular dysregulation, comprising:
determining whether a patient has a chronic medical condition and has one or more common symptoms based on a personal and medical history collected during a patient encounter, and if so,
treating the patient's common symptoms with vascular dysregulation techniques thereby improving the patient's chronic medical condition, wherein the vascular dysregulation techniques include eliminating environmental triggers, eliminating medical triggers and adjusting the patient's current medication profile to reduce or eliminate other prescribed medications, over-the-counter medications and to include a calcium channel blocker, a nasal anticholinergic or an antiemetic or a combination thereof.

14. The method of claim 13 wherein calcium channel blocker includes verapamil or cardizem, the nasal anticholinergic includes atrovent and the antimetic includes tigan, compazine, reglan or other antiemetics.

15. A method for altering medical thresholds for medical symptoms with vascular dysregulation, comprising:
determining one or more medical thresholds for one or more medical symptoms are determined for a patient;
identifying a plurality of types of triggers for the one or more medical thresholds;
reducing or eliminating the identified plurality of types of triggers;
adjusting the patient's current medication profile to reduce or eliminate prescribed medications, over-the-counter medications and to include a calcium channel blocker, a nasal anticholinergic or an antiemetic, or a combination thereof, thereby altering the one or more medical thresholds required to produce the one or more medical symptoms and reducing cumulative effects of the one or more medical symptoms.

16. The method of claim 15 wherein the plurality of triggers include environmental triggers and medical triggers.

17. The method of claim 15 wherein the step of identifying a plurality of types of triggers includes identifying one or more vascular dysregulation pathways.

18. The method of claim 17 wherein the one or more vascular dysregulation pathways include a nasal congestion, headache, asthma, fatigue, hypertension, obesity, attention deficit disorder (ADD)/attention-deficit hyperactivity disorder (ADHD), substance abuse, substance addiction, anxiety, depression, panic disorder, respiratory infections, common colds, downs syndrome, colic, insomnia, snoring, sleep apnea, gastroesophageal reflux disease (GERD), Raynaud's phenomenon, sudden infant death syndrome (SIDS):, stroke, seizures, chronic neck pain, premenstrual syndrome (PMS), chronic fatigue syndrome, Tourette's syndrome, Alzheimer's, or dementia vascular dysregulation pathway.

19. The method of claim 15 wherein calcium channel blocker includes verapamil or cardizem, the nasal anticholinergic includes atorvent and the antimetic includes tigan, compazine, reglan or other antiemetic.

20. The method of claim 15 wherein calcium channel blocker includes verapamil and cardizem.
